(19) United States
(12) Patent Application Publication
(10) Pub. No.: US 2026/0077198 A1
(43) Pub. Date: Mar. 19, 2026

(54) SYSTEMS, DEVICES, AND METHODS FOR DETERMINING TREATMENT FOR A NEUROLOGICAL DISORDER

(71) Applicant: NeuroPace, Inc., Mountain View, CA (US)

(72) Inventors: David A. Greene, Fort Wayne, IN (US); Brett M. Wingeier, San Francisco, CA (US)

(21) Appl. No.: 19/330,626

(22) Filed: Sep. 16, 2025

Related U.S. Application Data
(60) Provisional application No. 63/695,259, filed on Sep. 16, 2024.

(51) Int. Cl. A61N 1/36

(52) U.S. Cl. CPC A61N 1/36064; A61N 1/36139

(57) ABSTRACT

A system for selecting therapy parameters for a neurostimulation system associated with a brain includes a device configured to sense biosignal activity from the brain and an optimizer. The optimizer is configured to select a target region of the brain for neuromodulation therapy, which target region minimizes the difference between real biosignals captured by the device and modeled biosignals. The optimizer is further configured to determine stimulation settings for neuromodulation therapy, which stimulation settings result in a modeled electrical characteristic of stimulation at a target region that satisfies a criterion.

### CROSS-REFERENCE TO RELATED APPLICATIONS

This application claims the benefit of U.S. Provisional Application Ser. No. 63/695,259, entitled “Systems, Devices, and Methods for Determining Treatment for a Neurological Disorder” and filed on Sep. 16, 2024, which is expressly incorporated by reference herein in its entirety.

### TECHNICAL FIELD

The present disclosure relates generally to the determination of treatment for a neurological disorder, and more particularly, systems, devices, and methods for determination of treatment for a neurological disorder in which tissue imaging information and/or bioelectric signals are analyzed to deliver a spatial pattern of neurostimulation which is optimally coincident with a region of interest (for example, a seizure focus, seizure network, or portion of a seizure network) for treatment of the neurological disorder.

### BACKGROUND

Brain stimulation has emerged as a highly effective technique for treating a range of neurological disorders, including Parkinson's disease and epilepsy. Devices such as NeuroPace's RNS System for epilepsy and Medtronic's DBS for Parkinson's disease have received FDA approval and are now in clinical use. In addition to delivering therapeutic stimulation, some of these devices are capable of recording brain activity, which is particularly valuable for understanding the brain's response to stimulation. Analyzing brain activity before, during, and/or after pathological activity can provide crucial insights into how to optimize stimulation to effectively treat a disease or disorder.

In optimization of brain stimulation, it is often believed helpful to deliver a stimulation whose neuromodulatory effect on brain tissue is directed toward a particular target or region of interest, and is of similar spatial extent to the extent of the target or region of interest. This may avoid undesired side effects such as paresthesia from stimulating non-target regions, and may result in more efficient use of energy since little excess energy is expended stimulating non-target tissue.

One approach for such optimization is to model brain and surrounding tissue, for instance with finite element methods, using a one-size-fits-all model or universal brain atlas registered to a patient's imaging data, to estimate the spread of stimulation and compare it with a known or estimated stimulation target. For example, such an approach might assume that the brain is homogenous and that a voltage gradient of 0.2V/mm will activate tissue by stimulation. Using assumptions about conductivity and glial layer thickness, such an approach might then model the field geometry and define a region of ∇E>0.2 V/mm around the stimulation electrode(s) that defines the estimated volume of cortical activation. Some such approaches display to the user an estimate of the tissue being activated, such as by superimposing a brain atlas and axonal pathway model. Such approaches may also use look-up tables mapping various stimulation parameters to pre-defined field gradients or region boundaries, so the device displaying the estimate(s) can rapidly show estimates for various stimulation montages and amplitudes. Additionally, some existing approaches for optimization use patient-specific imaging data such as MRI or diffusion tensor imaging (DTI) data to generate a model of brain and surrounding tissue whose properties are customized to the individual patient.

Such methods currently known in the art may be effective for cases in which a target is determined anatomically, such as in neuromodulation for Parkinson's disease in which the dorsal region of the subthalamic nucleus is determined anatomically and targeted with electrical stimulation. However, current methods are not applicable to cases where a target is largely determined electrophysiologically. Examples include epilepsy in which the target is a seizure focus, seizure network, or portion of a network; cases such as treatment of Parkinson's disease by specifically targeting the portion of subthalamic nucleus displaying pathological beta rhythm or other signals; treatment of depression or other psychiatric diseases by targeting tissue displaying functional connectivity or synchrony between dorsolateral prefrontal cortex and subgenual cingulate; and treatment of Tourette syndrome by targeting tissue displaying low-frequency (e.g., delta, theta, and/or low alpha band) oscillations.

Given these limitations, there is a need for more advanced approaches using electrographic signals to estimate the region of interest for stimulation.

### SUMMARY

A system includes a database, an electrographic event module, and a probabilistic module. The database includes a plurality of records of biosignal activity of a brain sensed at different locations within or on the brain by different channels of an implanted medical device. The electrographic event module is configured to process the plurality of records to identify records corresponding to an event of interest, and for each identified record, to detect an event feature and a time of the event feature. The probabilistic module is configured to process the times of the event feature for the identified records and to determine for the different locations within or on the brain, a metric of times the event feature was first detected at that location.

An apparatus for selecting a therapy parameter of an implanted medical device includes an interface configured to receive a plurality of records of biosignal activity of a brain sensed at different locations within or on the brain by different channels of an implanted medical device, and a processor. The processor is configured to: process the plurality of records to identify records corresponding to an event of interest, and for each identified record, to detect an event feature and a time of the event feature; process the times of the event feature for the identified records and determine for the different locations within or on the brain, a metric of times the event feature was first detected at that location; and select, based on the metrics determined by the probabilistic module, one or both of a detection channel location from among the different locations for detecting by the implanted medical device and a stimulation channel location from among the different locations for stimulation therapy delivery by the implanted medical device.

A method of selecting one or more therapy parameters of a neurostimulation system includes identifying, from a set of EEG records captured through a plurality of channels of the neurostimulation system, those EEG records having a specified event; for the identified EEG records, determining the onset time of the specified event; calculating a probabilistic distribution of event onset timing across a plurality of channels; selecting at least one therapy setting based on the probabilistic distribution; programming the therapy setting into the neurostimulation system; and operating the neurostimulation system based on the therapy setting.

A system for selecting therapy parameters for a neurostimulation system associated with a brain includes a device configured to sense biosignal activity from the brain and an optimizer. The optimizer is configured to select a target region of the brain for neuromodulation therapy, which target region minimizes the difference between real biosignals captured by the device and modeled biosignals. The optimizer is further configured to determine stimulation settings for neuromodulation therapy, which stimulation settings result in a modeled electrical characteristic of stimulation at a target region that satisfies a criterion.

A method of selecting one or more settings for delivering stimulation therapy by a neurostimulation system, includes estimating a target region of a brain for the detection of an event of interest and the delivery of neurostimulation therapy; and estimating stimulation settings for the delivery of the neurostimulation therapy.

### BRIEF DESCRIPTION OF THE DRAWINGS

Various aspects of apparatuses and methods will now be presented in the detailed description by way of example, and not by way of limitation, with reference to the accompanying drawings, wherein:

FIG. 1 is a block diagram illustration of a system, including a neurostimulation system, an optimizer and other external equipment, that enable the selection and refinement of system parameters for the delivery of treatment of neurological disorders by the neurostimulation system.

FIG. 2 is a perspective, schematic illustration of a neurostimulation system implanted in a patient and configured to sense and record EEG records and provide such records as part of the system of FIG. 1.

FIG. 3 is a block diagram of the implanted neurostimulation system of FIG. 2, illustrating some of the functional subsystems of the system.

FIGS. 4A-4D are example visual representations of a physiological record in the form of an EEG record corresponding to electrical activity of the brain as recorded by an implanted neurostimulation system.

FIG. 5 is a block diagram illustration of an optimizer of FIG. 1.

FIG. 6 is an example visual representation of a physiological record in the form of an EEG record corresponding to electrical activity of the brain as recorded by an implanted neurostimulation system.

FIG. 7 is a graph of an electrographic event corresponding to ictal onsets as a function of time, across four channels of an implanted neurostimulation system.

FIG. 8 is a display of an implanted neurostimulation system including gray scale coded ictal onset information for a number of electrodes of the system.

FIG. 9 includes displays of the implanted neurostimulation system of FIG. 8, with the right-side image indicating a detection setting corresponding to a detection channel formed by two electrodes, and the left-side image indicating a stimulation setting corresponding to a subset of electrodes.

FIG. 10 is a flowchart of a method of determining one or both of a detection parameter and a stimulation parameter for a neurostimulation system based on electrographic data.

FIG. 11 is a flowchart of a method of determining one or both of a detection parameter and a stimulation parameter for a neurostimulation system based on electrographic data and imaging data.

FIG. 12 is an illustration of a data processing flow used in the method of FIG. 11 to select an initial estimated target region of the brain for one or both of the sensing of electrographic events and the delivery of neurostimulation therapy.

FIG. 13 is an illustration of a data processing flow used in the method of FIG. 11 to select a refined estimated target region of the brain for one or both of the sensing of electrographic events and the delivery of neurostimulation therapy.

FIG. 14 is an illustration of a graphical user interface and therapy guideline information used in the method of FIG. 11 to select initial estimated stimulation parameters for the delivery of neurostimulation therapy.

FIG. 15 is an illustration of a data processing flow used in the method of FIG. 11 to select refined estimated stimulation parameters for the delivery of neurostimulation therapy.

FIG. 16 is a schematic block diagram of an apparatus corresponding to the optimizer of FIG. 1.

### DETAILED DESCRIPTION

Systems, devices, and methods disclosed herein use probabilistic or similar analyses of biosignals to automatically determine parameters for treatment of neurological disorders by a neurostimulation system. The automatically determined parameters include, for example, a target region for the delivery of neuromodulation therapy, accompanied by modeling of a region of neuromodulation, and stimulation parameters, and/or detection such that the modeled region of neuromodulation optimally covers the target region. This offers a novel solution for treatment of disorders where a target for neuromodulation is partially or largely determined electrophysiologically.

Systems, devices, and methods disclosed herein provide visualization of seizure propagation in patients with chronically implanted medical devices with a plurality of leads and sensing/detection channels at multiple brain locations, which visualization is based on probabilistic or similar analyses of biosignals corresponding to electrical activity of the brain, referred to herein as EEGs or electrocorticograms (ECoGs).

Systems, devices, and methods disclosed herein provide enhanced configuration and/or optimization of detection settings and/or stimulation settings for chronically implanted medical devices with a plurality of leads and sensing/detection channels at multiple brain locations, including automating the determination of settings, and automating the implementation of the determined setting in the implanted medical devices.

In patients with implanted medical devices (IMD) for treating epilepsy, many ictal events may be recorded over extended time periods. Ictal events may be electrographic seizures; some ictal events may be clinical seizures as well as electrographic seizures. Some ictal events may be stereotypical with onset locations in the same locations consistently, but the distribution of the propagation can only be ascertained after collection of a significant number of events, and patients also may have multiple seizure types that begin and propagate through different networks. The same is true of sub-ictal events, such as but not limited to epileptiform activity, epileptiform spikes, spike-wave complexes, high frequency low amplitude bursts, or other forms of epileptiform activity.

Embodiments disclosed herein that refer to the processing or use of biosignals of seizure or ictal events to determine parameters for treatment of neurological disorders may additionally or alternatively process or use biosignals of sub-ictal events, other episodic electrophysiological events characteristic of a neurological disease, or evoked potentials unless otherwise specified. Embodiments disclosed herein may additionally or alternatively process or use characteristics of biosignals of non-episodic electrophysiological events, for instance by using phase or instantaneous phase of an ongoing waveform or frequency component thereof as a proxy for arrival time; in such embodiments, for example, channels which sense the waveform at an earlier phase are treated as channels detecting or sensing the activity earlier, and channels which sense the waveform at a later phase are treated as channels detecting or sensing the activity later.

### Overview of System

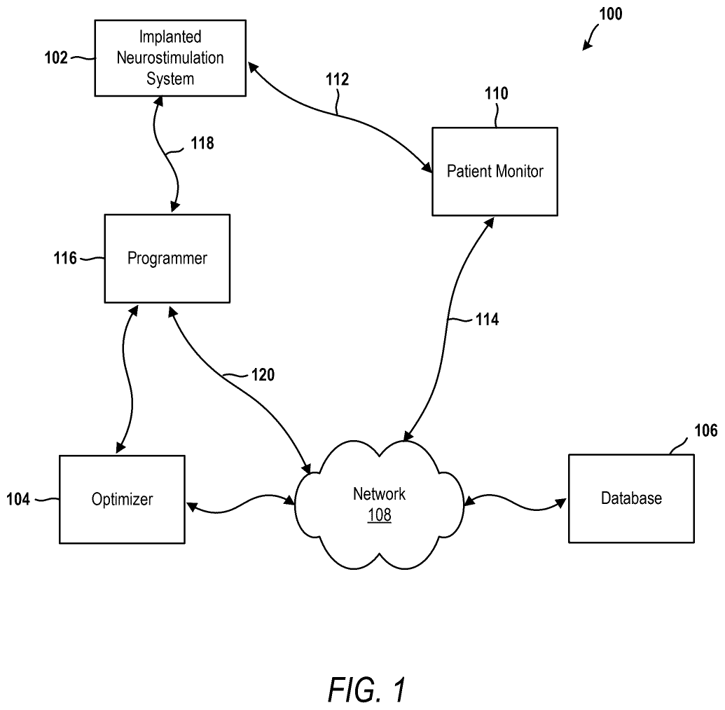

FIG. 1 is a block diagram illustration of a system 100 in which probabilistic or similar analyses of biosignals are used to automatically determine system parameters for treatment of neurological disorders by a neurostimulation system. The automatically determined system parameters include, for example, a target region for the delivery of neuromodulation therapy, accompanied by modeling of a region of neuromodulation, and stimulation parameters (e.g., amplitude and electrode montage for stimulation delivery) and/or detection parameters (e.g., electrode montage for sensing and detection of events). The system includes an implanted neurostimulation system 102, an optimizer 104, and a database 106, each configured to provide and/or obtain a patient's physiological information over a network 108. While shown as an independent component, the optimizer 104 may be part of a programmer 116 or a patient monitor 110, or may be co-located with the database 106.

Physiological information corresponding to EEG records may be captured by the implanted neurostimulation system 102. As described later below, these EEG records may correspond to digitally recorded time series samples of electrocorticographic activity (e.g., a time series waveform). These EEG records may also be in another form or format derived from the time series samples. For example, an EEG record may be a spectrogram image or a time series waveform image of the brain electrical activity. (It will be appreciated that any time-series EEG can be represented as a spectrogram.) Alternatively, time-series waveforms may be directly used.

Other types of physiological information, e.g., pH levels, blood oxygen levels, neurotransmitters concentrations, heart rate, blood pressure, blood glucose levels, hormone levels, sleep states, posture, etc., may be captured and preserved by an implanted neurostimulation system 102 as physiological records. Collectively, the EEG records and other physiological records preserved by an implanted neurostimulation system 102 are part of a dataset for the patient in which the device is implanted. Non-physiological information may additionally form part of the dataset and may include records or files of patient demographics (e.g., age, gender), patient drug regimen (e.g., type of drug, dose, and time of day of dose), and patient clinical outcomes, such as the rate of electrographic seizure detection and electrographic seizure onset (e.g., as detected and recorded by the implanted neurostimulation system), or the rate of clinical seizures (e.g., as reported in a seizure diary or detected based on accelerometer recordings).

The neurostimulation system 102 includes implantable components, namely, an active medical device or neurostimulator, and one or more electrode-bearing leads. The electrodes are configured to rest in or on neural tissue in the patient's brain when the leads are implanted. The neurostimulator may be configured to be implanted in or on the patient's cranium or elsewhere in the patient (e.g., pectorally). Once the neurostimulator is implanted, a proximal end of each lead is connected to the neurostimulator. The combination of the active implanted medical device and the implanted lead(s) is configurable to sense physiological signals from the brain and process and store records of the sensed signals. In this example, the physiological signals the electrodes sense and transmit through the lead(s) to the neurostimulator are electrocorticographic signals. The neurostimulator is configured to record samples or segments of the sensed EEGs, and to store them in a memory.

A neurostimulation system 102 may capture different data types based on EEG signals. Data types may be captured at different time scales. Some examples of data types captured by a neurostimulation system 102 include: (1) continuous recordings (EEG records) of raw brain data at a certain sampling rate such as 1000, 500 or 250 Hz, (2) continuous measures of derived brain data such as spectral power in certain frequency bands (example 1-4 Hz band, 4-8 Hz band, 8-2 Hz band, 12 -25 Hz band, 25-50 Hz band, 50 -90 Hz band and so on) computed in small moving and overlapping time windows such as 128, 256 or 512 milliseconds; (3) counts of abnormal events in bins of varying durations such as minutes, days or hours; (4) sampled raw time series or derived brain data that are saved at random time points, specific time points (preprogrammed by a physician for example) or are sampled in response to a trigger such as detection of abnormal events in brain or when a patient swipes a magnet over the neurostimulator; and (5) patient reports of outcomes. These are almost always not continuous and only intermittently available.

A neurostimulation system 102 may also be configured to sense and record other types of physiological signals besides EEG signals. To this end, the neurostimulation system 102 may include a lead as disclosed in U.S. Pat. No. 10,123,717, entitled Multimodal Brain Sensing Lead, which is herein incorporated by reference. Such a multimodal brain sensing lead may include: (1) macroelectrodes; (2) microelectrodes; (3) light emitters; and (4) photodetectors. Different sensing modalities of the implanted neurostimulation system 102 use the different transducers as follows: (1) neuronal field potential measurements are made using macroelectrodes; (2) neuronal single unit activity measurements are made using microelectrodes; (3) neuronal multi-unit activity measurements are also made using microelectrodes; (4) rheoencephalography measurements are made using macroelectrodes; (5) neurochemical and pharmaceutical voltammetric measurements are made using both macroelectrodes and microelectrodes; (6) optical blood flow and volume measurements are made using light emitters and photodetectors; and (7) optical blood oxygenation measurements are also made using light emitters and photodetectors.

Configured as such, the neurostimulation system 102 may sense and record signals indicative of blood oxygen level and blood volume in neural tissue, and signals indicative of chemical concentrations and neurotransmitter concentrations in neural tissue. From these signals, the neurostimulation system 102 may derive other physiological information. For example, blood flow, blood oxygenation, blood pressure, heart rate, and breathing rate may be estimated from blood oxygen and blood volume measurements, while pH levels and blood glucose levels may be derived from chemical concentrations and neurotransmitter concentrations.

The neurostimulation system 102 may also include one or more electrodes configured to sense electrical cardiac activity indicative of heart rate, a pressure sensor configured to provide signals indicative of blood pressure, and/or an accelerometer configured to provide motion signals indicative of motion and the position of the patient. From these accelerometer signals, the implanted neurostimulation system 102 may derive other physiological information corresponding to clinical seizures, patient posture, and sleep state.

Other types of physiological information may be obtained and stored by 102 of the neurostimulation system 102 from sources independent of the neurostimulation system. For example, an external wearable device, e.g., patch, may include a sensor configured to sense and track cortisol levels, i.e., stress hormones, in sweat, while an external wearable device, e.g., watch, may include sensor(s) configured to sense blood pressure, blood oxygenation, heart rate, skin temperature, galvanic skin response, and/or accelerometry. The physiological information from these external devices may be transmitted to the implanted neurostimulation system 102 for inclusion in the patient's dataset; alternatively, the physiological information may be transmitted separately to the database 106 by the external wearable device and thereafter included into the patient's dataset, or the external wearable device may communicate with the implanted neurostimulation system 102 to retrieve data collected by the implanted system and transmit it to the database 106.

Records of physiological information may be generated by the neurostimulation system 102 based on an occurrence of an event or trigger. To this end, a neurostimulation system 102 can be configured to create an EEG record of a sensed EEG when an event the system is programmed to detect is detected. For example, the neurostimulation system 102 may be configured to detect an event corresponding to an electrographic seizure or the onset of an electrographic seizure from a sensed EEG, and to create an EEG record of the corresponding EEG signal spanning the time period 60 seconds before the event was detected and 30 seconds thereafter. The neurostimulation system 102 can also be programmed to create an EEG record of a sensed EEG at certain times of day (e.g., at noon and at midnight). These are sometimes referred to as “scheduled EEGs.” In addition, the neurostimulation system 102 may be configured to store an EEG record upon some other trigger, such as when the patient swipes a magnet over the location on the patient's body at which the neurostimulator is implanted (the patient might be instructed to do this whenever he or she thinks a seizure is coming on).

The neurostimulation system 102 can also be programmed to designate EEG records based on the event that triggered its recording and to include that designation in the EEG record. For example, EEG records resulting from the detection of abnormal electrical activity, e.g., an electrographic seizure or the onset of an electrographic seizure, may be marked as such, while EEG records that do not reflect abnormal activity may be designated as baseline EEG records. Thus, for a given patient, a dataset may contain EEG records corresponding to what is happening in the patient's brain during and around when an event occurs, scheduled EEG records acquired at a particular time, and EEG records stored by the neurostimulator when a patient triggers storage with a magnet. Some of these EEG records, especially the ones recorded at the time of an event or when triggered by a magnet swipe, may reflect the patient's electrographic seizures. The dataset may include information or a data type about whatever triggered the neurostimulator to store a given EEG, such as the type of electrographic pattern detected (e.g., Pattern “A” or Pattern “B”), a patient triggered event (e.g., a magnet swipe), or the time of day (e.g., scheduled EEG).

Typically, some sort of linkage or mapping among the various types of physiological information is provided in a dataset. To this end, each record may have one or more associated tags or parameters. For example, physiological records may have a time stamp that allows a set of physiological records at a given point in time to be located for processing. Physiological records may have a tag that indicates the basis, e.g., seizure detection, magnet swipe, scheduled time of day, for preserving the record. These tags allow a set of physiological records to be selected for processing based on a single criterion or a combination of criteria. Other tags may include day of capture, area of the brain at which the electrical activity was captured, basis for record creation (e.g., seizure detection, scheduled, patient initiated), characteristics of the record (e.g., power spectral density of EEG signal prior to stimulation).

Once created by a neurostimulation system 102, physiological records stored in the system can be relayed elsewhere, such as to an external component like the database 106 either directly or through an interim external component. For example, the patient monitor 110 can be used with an accessory (not shown) to establish a communications link 112 with the implanted neurostimulator (e.g., a short-range telemetry link), which allows records stored on the neurostimulator to be transmitted to the patient monitor 110. Once on the patient monitor, the physiological records can be transmitted to the database 106 via the network 108 over a communication link 114 (which may comprise a physical, WiFi, or cellular internet transmission).

Alternatively, the clinician may be provided with an external component, such as a programmer 116 that, like the patient monitor 110, is configured to establish a communications link 118 with the implanted neurostimulator. The clinician can use the programmer to adjust the programmable parameters of the neurostimulator (e.g., the parameters that govern the electrical stimulation waveform that is used for therapy). The programmer 116 is able to specify and set variable parameters in the implanted neurostimulator 202 (e.g., detection parameter sets and stimulation parameter sets) to adapt the function of the device to meet the patient's needs, upload or receive data from the neurostimulation system to the programmer, download or transmit program code and other information from the programmer to the neurostimulator, or command the neurostimulator to perform specific actions or change modes as desired by a physician operating the programmer.

The programmer 116 also may be used to display the real time EEG signals being sensed by the electrodes from the patient and to store them on the programmer. It also can be used like the patient monitor 110 to acquire physiological records that have been stored by the neurostimulator since the last time the neurostimulator was “interrogated” for those records by either a patient monitor 110 or programmer. As is the case with a patient monitor 110, once physiological records are stored on a programmer 116, they can be transmitted via a communication link 120 and the network 108 to other components of the system 100, such as the database 106 and/or the optimizer 104 (either directly or via the database 106).

A neurostimulation system 102 may be configured to deliver electrical stimulation therapy in response to “events” that the neurostimulator is configured to detect. An event may be defined for the neurostimulator by setting the values of programmable detection parameters such that when a pattern corresponding to a pattern defined by the detection parameters occurs in the monitored EEG signals, the occurrence of that pattern will be detected as an event. Other implantable neurostimulation systems that might be used in the subject system may not have this feature of responsive neurostimulation at all or may not have it enabled.

While FIG. 1 illustrates a single implanted neurostimulation system 102 and patient monitor 110 and programmer 116, numerous neurostimulation systems implanted across a patient population may access the network 108 to provide patient physiological records and patient information to the optimizer 104 and the database 106. Accordingly, the system 100 can provide access to large numbers (e.g., thousands, millions, or more) of patient EEG records.

### Overview of Implanted Neurostimulation System

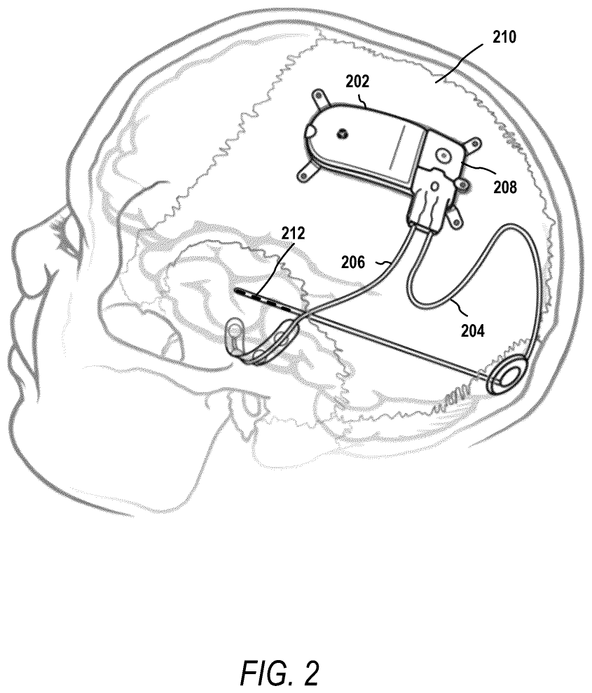

FIG. 2 is an illustration of the implanted neurostimulation system including a neurostimulator 202 and two electrode-bearing brain leads 204, 206, implanted in a patient. The system is configured to sense and record electrical brain activity and other physiological information and provide such records as part of the system of FIG. 1. In other embodiments, the implanted neurostimulation system might include a different number of electrode-bearing brain leads, such as one lead, three leads, four leads, or more.

The neurostimulator 202 includes a lead connector 208 adapted to receive one or more of the brain leads, such as a deep brain or depth lead 204 and a cortical strip lead 206. The depth lead is implanted so that a distal end of it is situated within the patient's neural tissue, whereas the cortical strip lead is implanted under the dura mater so that a distal end of it rests on a surface of the brain. The lead connector 208 acts to physically secure the brain leads 204, 206 to the neurostimulator 202, and facilitates electrical connection to conductors in the brain leads 204, 206 coupling one or more electrodes at or near a distal end of the lead to circuitry within the neurostimulator 202.

The proximal portion of the deep brain lead 204 is generally situated on the outer surface of the cranium 210 (and under the patient's scalp), while the distal portion of the lead enters the cranium and is coupled to at least one depth electrode 212 implanted in a desired location in the patient's brain. The proximal portion of the cortical lead 206 is generally situated on the outer surface of the cranium 210 (and under the patient's scalp), while the distal portion of the lead enters the cranium. The distal portion of the cortical lead 206 includes at least one cortical electrode (not visible) implanted in a desired location on the patient's brain.

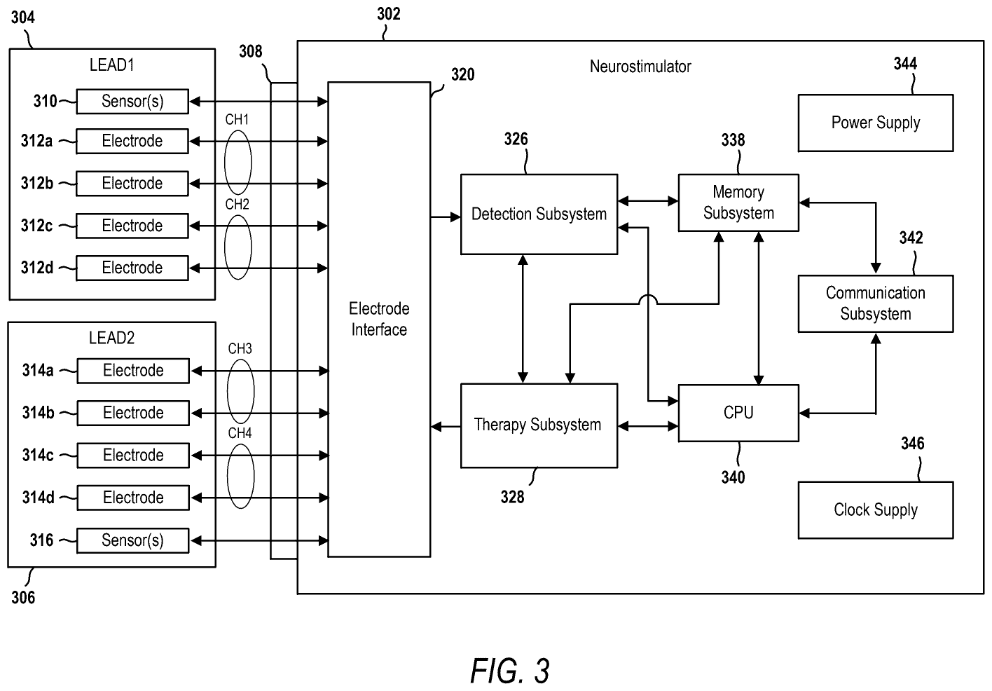

FIG. 3 is a block diagram of the implanted neurostimulation system of FIG. 2. The system may be configured to sense electrical brain activity, detect events in accordance with a set of detection parameters, deliver electrical neurostimulation to the brain in accordance with a set of stimulation parameters, and store records of electrical brain activity and other physiological information for transmission to the database 106 of the system of FIG. 1.

The neurostimulator 302 includes a lead connector 308 adapted to receive a connector end of each brain lead 304, 306, to thereby electrically couple each lead and its associated electrodes 312a-d, 314a-d with the neurostimulator. The neurostimulator 302 may configure an electrode 312a-d, 314a-d as either a sensor (for purposes of sensing electrical activity of the brain) or a stimulator (for purposes of delivering therapy to the patient in the form of electrical stimulation) or both. In other embodiments, each lead may have a different number of electrodes, such as eight electrodes, or may be configured as a cortical strip, grid, or other form factor.

The electrodes 312a-d, 314a-d are connected to an electrode interface 320. The electrode interface 320 can select each electrode 312a-d, 314a-d as required for sensing and stimulation. The electrode interface 320 may also provide other features, capabilities, or aspects, including but not limited to amplification, isolation, and charge-balancing functions, that are required for a proper interface with neurological tissue. The electrode interface 320 is coupled to a detection subsystem 326, which is configured to process electrical activity of the brain sensed through the electrode 312a-d, 314a-d. The electrode interface 320 may also be coupled to a therapy subsystem 328, which is configured to deliver therapy to the patient through the electrode 312a-d, 314a-d in the form of electrical stimulation.

One or both of the brain leads 304, 306 may have one or more physiological sensors 310, 316 that enable the capture and recording of other types of physiological information, e.g., pH levels, blood oxygen levels, neurotransmitters concentrations, heart rate, blood pressure, blood glucose levels, hormone levels, sleep states, posture, etc. To this end, one or both of the brain leads 304, 306 may be configured as disclosed in U.S. Pat. No. 10,123,717, entitled Multimodal Brain Sensing Lead, which is herein incorporated by reference, and the one or more physiological sensors 310, 316 may correspond to different transducers, e.g., macroelectrodes, microelectrodes, light emitters, and photodetectors that enable different sensing modalities.

The neurostimulator 302 includes a memory subsystem 338 and a central processing unit (CPU) 340, which can take the form of a microcontroller. The memory subsystem 338 is coupled to the detection subsystem 326, and may receive and store records of data representative of sensed electrographic signals for transmission to the system of FIG. 1. The memory subsystem 338 is also coupled to the therapy subsystem 328 and the CPU 340. In addition to the memory subsystem 338, the CPU 340 is also connected to the detection subsystem 326 and the therapy subsystem 328 for direct control of those subsystems.

The neurostimulator 302 also includes a communication subsystem 342. The communication subsystem 342 enables communication between the neurostimulator 302 and an external device, such as a programmer 116 or patient monitor 110, through a wireless communication link. As described above with reference to FIG. 1, the programmer 116 allows a clinician to read out records of patient data, as well as ancillary information associated with those records. The neurostimulator 302 also includes a power supply 344 and a clock supply 346. The power supply 344 supplies the voltages and currents necessary for each of the other subsystems. The clock supply 346 supplies substantially all the other subsystems with any clock and timing signals necessary for their operation.

### Physiological Information

With respect to physiological information, a dataset may include records or files of physiological information corresponding to electrical activity of the brain. Hereinafter, electrical activity of the brain is referred to as an “EEG,” the digital representation, i.e., stored data bits, of electrical activity of the brain is referred to as “EEG data,” and a physiological record corresponding to electrical activity of a patient's brain is referred to as an “EEG record.” It will be understood that EEG includes electrical activity sensed directly from the neural tissue, which sometimes is referred to as electrocorticographic activity, an electrocorticogram, or “ECoG,” although such electrical activity also may include activity sensed from neural tissue other than cortex, for example thalamic or other subcortical neural tissue.

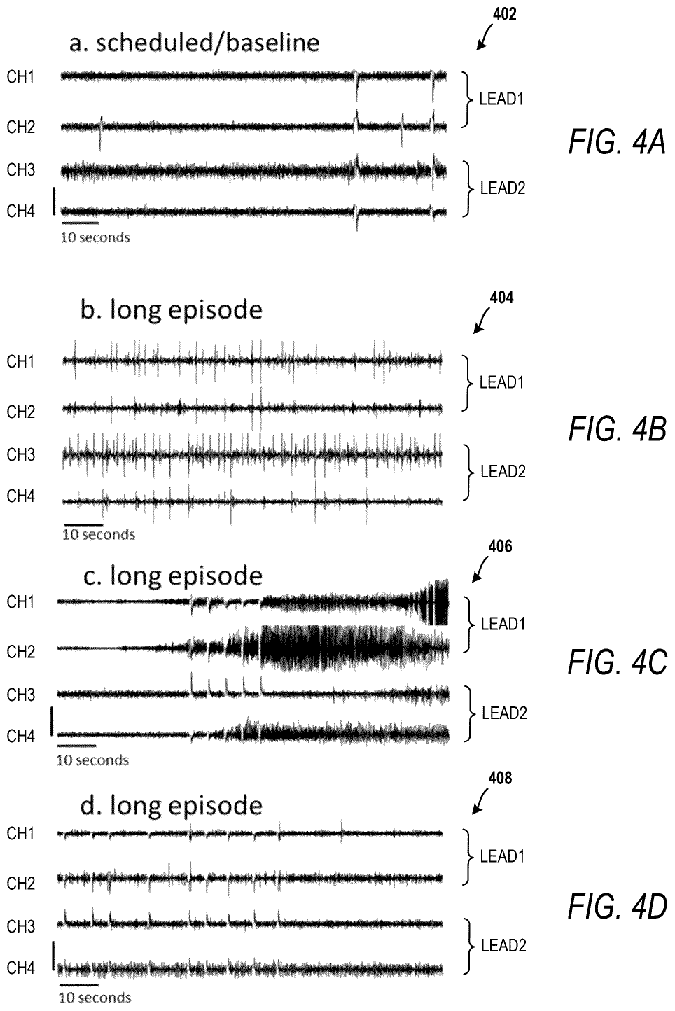

An EEG record corresponding to electrical activity of a patient's brain may be visualized in the form of a time series waveform image. For example, with reference to FIGS. 4A-4D, an EEG record 402, 404, 406, 408 may consist of four channels (CH1, CH2, CH3, CH4) of EEG data, each visualized as a separate time series waveform, where two channels (CH1 and CH2) are associated with a first lead (LEAD1), and two channels (CH3 and CH4) are associated with a second lead (LEAD2). These four separate time series waveforms (and their corresponding EEG data) collectively represent the EEG record 402, 404, 406, 408. In some embodiments, the systems and methods disclosed herein process EEG records 402, 404, 406, 408 on a per channel basis. Accordingly, each channel time series waveform (and it corresponding EEG data) may also be referred to herein as an EEG record.

The EEC records illustrated in FIGS. 4A-4D are examples of types of EEG data captured by the system in one example patient. As described above with reference to FIG. 3, the neurostimulator can be connected to up to two leads. The leads may be either strip or depth leads. EEG files captured by the neurostimulator typically contain four channels of data. Channels 1 and 2 typically belong to lead 1, and channels 3 and 4 typically belong to lead 2.

The two main types of EEG records captured and stored by the device include scheduled EEG records (FIG. 4A), and long episode EEG records (FIGS. 4B-4D). Scheduled EEG records generally contain background non-seizure activity, and its storage is triggered by time of day. Long episode EEG records contain long trains of abnormal EEG activity which can sometimes be electrographic seizures. Automatic capture and storage of long episodes EEG records is triggered when long trains of abnormal events are sensed by the neurostimulator.

While the methods and systems disclosed herein are primarily described with reference to EEG records in the form of time series waveform images, other forms of EEG records may be used. For example, EEG records in the form of spectrograms may be processed by the methods and systems. Furthermore, while the methods and systems disclosed herein are primarily described with reference to records comprising electrical activity of the brain, it will be appreciated that other physiological information and non-physiological information may be processed.

### Optimizer

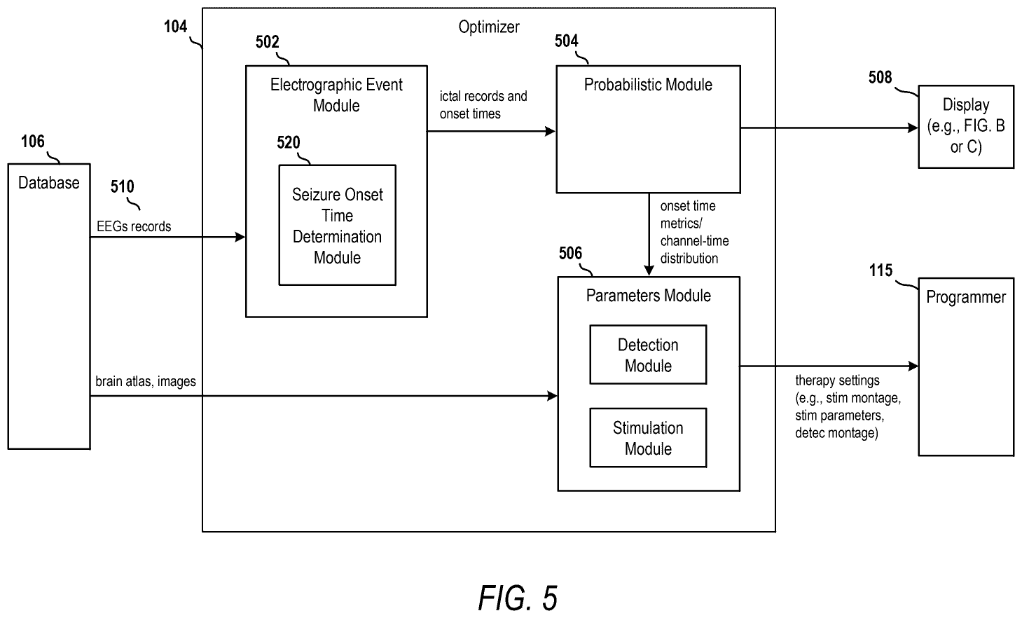

With reference to FIG. 5, in some embodiments the optimizer 104 includes an electrographic event module 502, a probabilistic module 504, and a parameters module 506, each configured to process records of biosignal activity and/or information derived therefrom. The optimizer 104 is configured to interface with the database 106 of FIG. 1 for purposes of receiving records of biosignal activity of a brain and other information, such as brain atlases and brain images. The optimizer 104 can also interface with a programmer 116 or patient monitor 110 for the purpose of programming an IMD. The optimizer 104 also interfaces with a display 508 for purposes of displaying records of biosignal activity and/or information derived therefrom.

The records of biosignal activity can be EEG records that include a plurality of channel records, where each channel record corresponds to biosignal activity sensed at a different location within or on the brain by a channel of an implanted medical device. In some embodiments the electrographic event module 502 is configured to process the plurality of the records to identify records corresponding to event (e.g., ictal) records and for each event record, to detect an event feature (e.g., seizure onset) and a time of the event feature for one or more of the plurality of channel records. The electrographic event module 502 may correspond to a machine-learned model trained in accordance with one of the models or a combination of models described in Barry W, Arcot Desai S, Tcheng TK and Morrell MJ (2021), A High Accuracy Electrographic Seizure Classifier Trained Using Semi-Supervised Labeling Applied to a Large Spectrogram Dataset. Front. Neurosci. June 2021, Vol. 15, Art. 667373, which is incorporated by reference. The electrographic event module 502 may also or additionally correspond to other models or methods such as, without limitation, vision transformer models, convolutional neural networks, recurrent neural networks, random forest classifiers, half-wave detectors, area-under-the-curve detectors, line length detectors, bandpass filtering, or other methods for event detection known to those skilled in the art.

In some embodiments the electrographic event module 502 includes a seizure onset time determination module 520. The seizure onset time determination module 520 may be a deep learning model configured to identify a seizure onset on individual channel EEG records and to output a corresponding seizure onset time. In some embodiments, the seizure onset time determination module 520 comprises a vision transformer based architecture. In this architecture, a large dataset of EEG records is manually labeled for seizure onset times on individual EEG channels and a vision transformer model is trained on the labeled dataset. The trained model is applied to EEG records to identify event features, e.g., seizure onsets, and provide times of event features. In other embodiments, the seizure onset time determination module 520 may be implemented using different methods, such as attention models (or other sequence based models) or signal processing algorithms.

The probabilistic module 504 is configured to process the times of the event onset for the ictal records and to convey (e.g., graphically display) metrics or information indicative of locations within or on the brain where the event onset was detected and for each location, the number of times the event onset was first detected at that location.

The parameters module 506 is configured to select parameters for treatment of neurological disorders by the implanted neurostimulation system 102. The automatically determined parameters can include one or more of: stimulation parameters, a target region for the delivery of stimulation therapy, and detection parameters (e.g., electrode montage for sensing and detection of events).

Stimulation parameters or settings may include but are not limited to amplitude, pulse width, and/or stimulation montage. Stimulation montage refers to one or more settings which determine the electrode path(s) that stimulation is delivered through. In its most general form, stimulation montage is a specification of one or more electrode groups, where an electrode group comprises a set of one or more electrodes which serve jointly as the cathode for stimulation and a set of one or more electrodes, distinct from the cathode set, which serve jointly as the anode for stimulation. In some embodiments, electrodes corresponding to the channel on which an event feature, e.g., seizure onset, was detected most frequently and/or earliest are chosen for delivery of stimulation therapy. Additionally, electrodes corresponding to channels having typically-later detection of an event may be chosen for delivery of additional stimulation therapy, for example in cases where the implantable neurostimulator system 102 determines that a seizure event is prolonged past the initial seizure onset. Additionally, stimulation settings may include multiple amplitude and/or pulse width settings. For example, in an embodiment where the implantable neurostimulator 102 has multiple independent current sources available, the settings can include one amplitude setting for each electrode group in a stimulation montage.

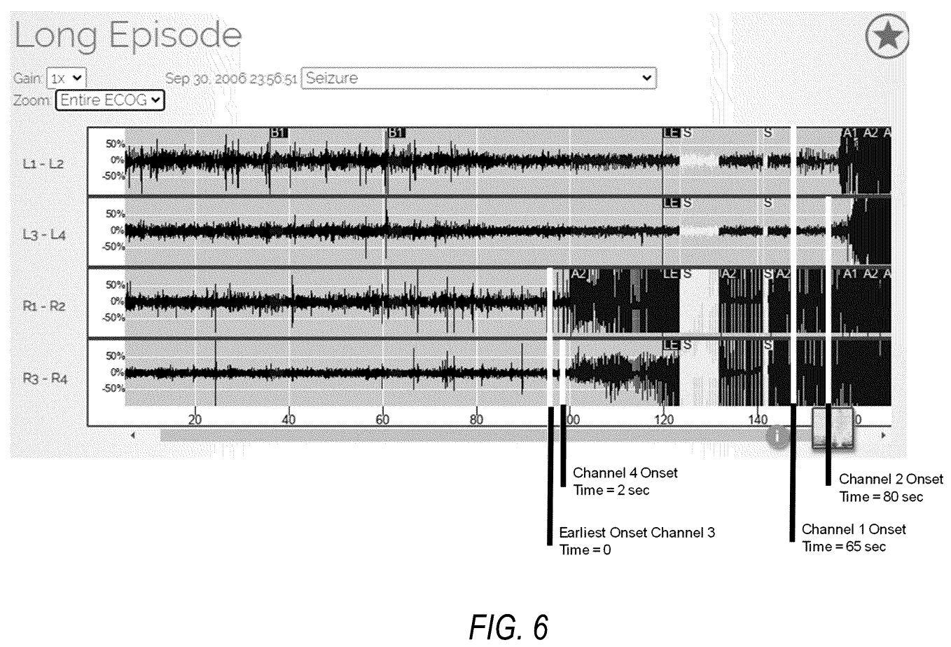

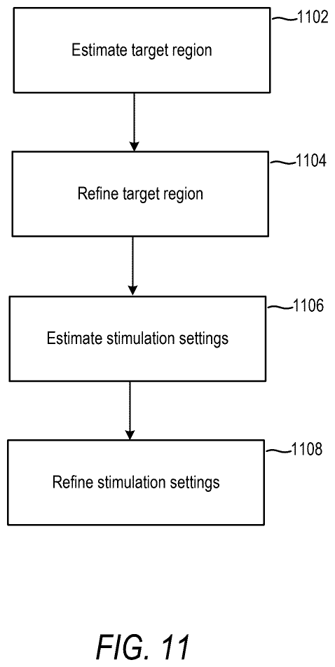

In some embodiments as disclosed below with reference to FIGS. 6-10, parameter selection by the parameters module 506 is based on event characteristics and/or event onsets provided by the probabilistic module 504. In other embodiments as disclosed below with reference to FIGS. 11-15, parameter selection by the parameters module 506 is based on event characteristics and/or event onsets provided by the probabilistic module 504 and additional information including electrographic signals, brain atlases, and/or brain images obtained from the database 106.

FIG. 6 is an example four-channel EEG record of a seizure event captured by an IMD with four sensing channels derived from two physically distinct leads that are implanted in two different locations in the brain. EEG records having more than four channels, e.g., eight sensing channels provided by a four lead IMD, may also be processed as described herein. EEG records having fewer than four channels, e.g., three channels, may also be processed as described herein. Additionally, multiple channels which are stored as more than one record but which contain data recorded simultaneously may be processed as described herein. However, for simplicity the description going forward focuses on the use of four-channel EEGs.

Using the machine learning methods disclosed in U.S. Patent Application Publication No. 2023/0172529, and in Barry W, Arcot Desai S, Tcheng TK and Morrell MJ (2021), A High Accuracy Electrographic Seizure Classifier Trained Using Semi-Supervised Labeling Applied to a Large Spectrogram Dataset. Front. Neurosci. June 2021, Vol. 15, Art. 667373, which are incorporated herein by reference, EEGs that are ictal can be classified as such, and the ictal onset time, also referred to herein as seizure onset time, for each channel can be determined. Other classification methods may also be used to determine the presence and onset time of an ictal, sub-ictal, or other electrographic event. These methods include but are not limited to vision transformer models, convolutional neural networks, recurrent neural networks, random forest classifiers, half-wave detectors, area-under-the-curve detectors, line length detectors, bandpass filtering, etc. Additionally, in embodiments operating on non-episodic waveforms, the presence of the waveform may be determined by classification methods such as those mentioned here, while an arbitrary time point such as the start of the record is chosen to serve as “onset time” to allow calculation of relative phase offsets with respect to this time point. In the EEG record shown in FIG. 6, the time of ictal onset is indicated for each channel relative to the earliest ictal onset which is on channel 3 (R1-R2 in the figure).

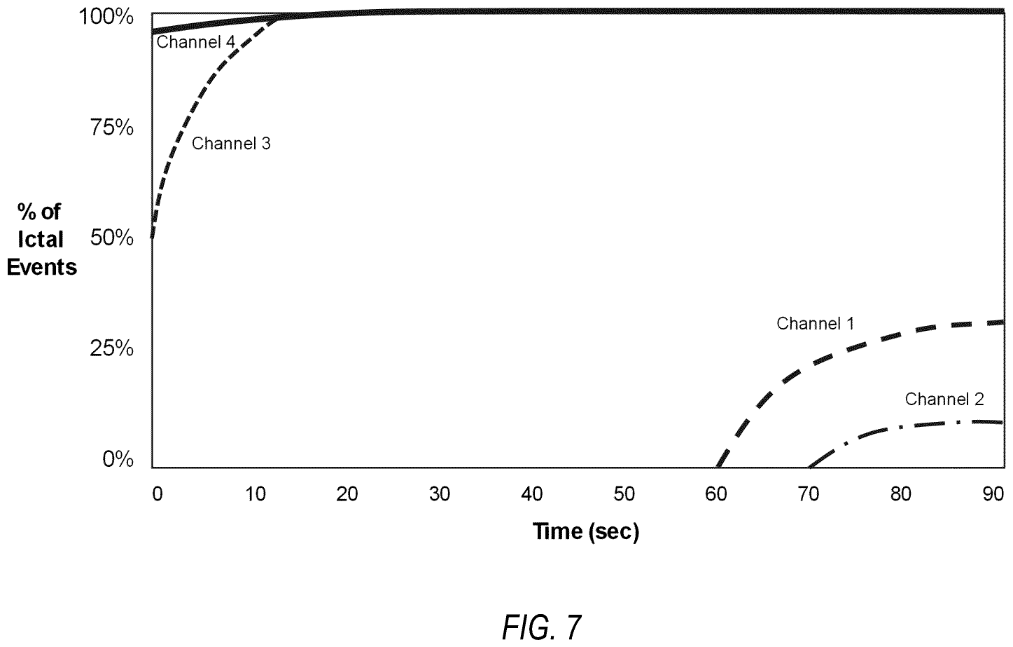

In accordance with embodiments disclosed herein and with reference to FIG. 7, a plurality, e.g., 100s or 1000s, of events for a patient are processed by the probabilistic module 504 to determine a distribution of an event feature, e.g., earliest onset time, on each channel and to produce timing distribution curves that show the channels with the earliest onset time and the time delays of propagation across the plurality of leads and channels. Alternatively, in embodiments operating on non-episodic waveforms, a plurality of waveform segments are processed to determine a distribution of the relative phase observed on each channel with respect to, e.g., the phase observed at a reference channel or an arbitrary time point serving as “onset time.”

In the example distribution shown in FIG. 7, the following data outcomes are determined by the automated process:
A) 95% of the time the event onset appears on Channel 4 first.
B) Approximately 50% of the time in the events the onset is simultaneous in channel 3.
C) Event onset is never observed first on channel 1 or 2.
D) Event onset often does not propagate (spread) to channels 1 or 2, and when it does spread to these channels, there is a significant delay of >60 seconds for channel 1 and >70 seconds for channel 2.

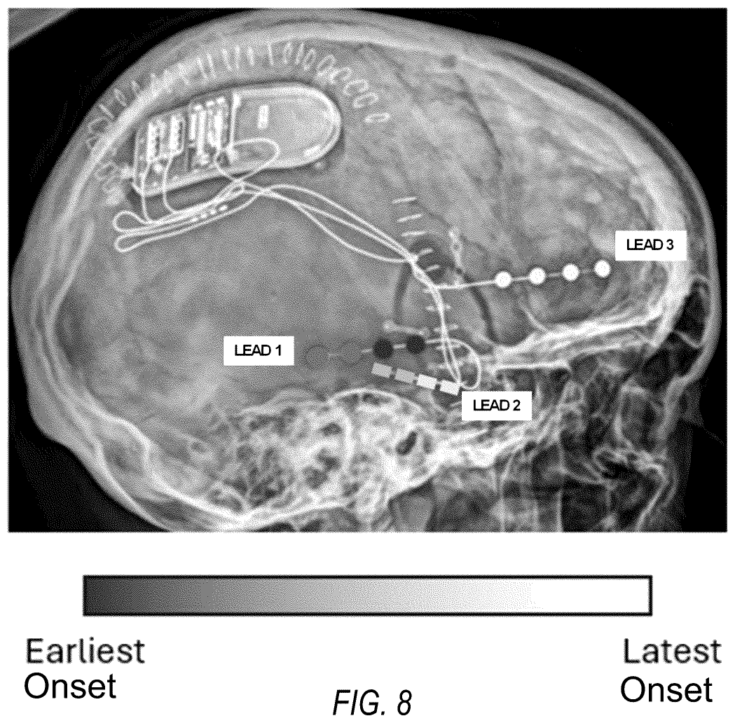

With reference to FIG. 8, using these data, the probabilistic nature of the spread of epileptiform activity in an implantable system with a plurality of electrodes could be assessed over long periods and over many events in an automated manner to present clinicians with graphical representations that guide or automate the programming of device setting, including either or both of detection settings and stimulation settings. In the representative example shown in FIG. 8, color intensity (shade of gray) is used to indicate event onset timing on a plurality of leads of an implanted medical device having 3 leads, each with four electrodes. While the example shown in FIG. 8 assumes a configuration in which each of four sensing channels monitors the bipolar potential difference across two adjacent electrodes on a lead, embodiments may also include other sensing montages; for example, “pseudo-monopolar” configurations where each sensing channel monitors the bipolar potential difference between an electrode on a lead and a distance reference such as the neurostimulator can.

These data outcomes from the probabilistic module 504 are used by the parameters module 506 to select from among a plurality of detection channels, the detection channel that is estimated to provide the earliest detection of an event. The data outcomes from the probabilistic module 504 can also be used by the parameters module 506 to determine which electrodes are involved in the ictal process and based on that determination, to select from among a plurality of stimulation channels, the stimulation channel through which stimulation therapy is to be delivered, while possibly avoiding side effects by avoiding stimulation on uninvolved electrodes.

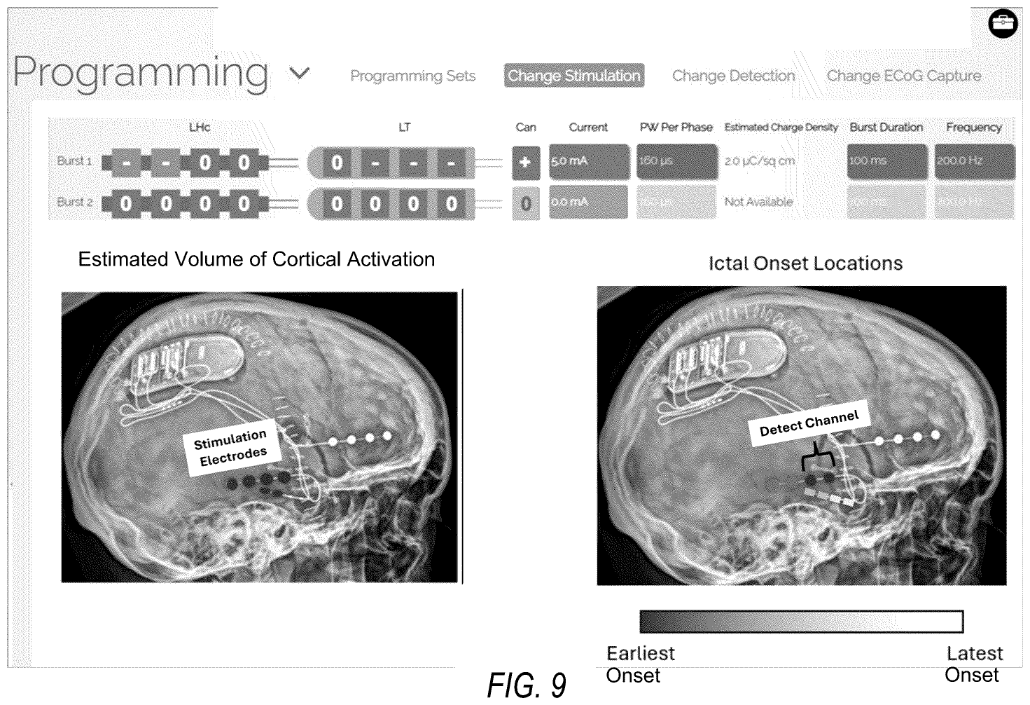

For example, with reference to FIG. 9, based on the data of FIG. 8 the parameters module 506 may select: 1) a detection channel formed by the darkest electrodes (the two indicated electrodes) in right side image FIG. 9, and 2) one or more simulation channels formed by a subset of the electrodes shown in dark gray in the left side image of FIG. 9. Regarding stimulation, in one example, the IMD can be programmed to stimulate on three simulation channels, wherein two channels are formed by the four electrodes on a first lead and the third channel is formed by two electrodes on a second lead. In some embodiments, the selections of the parameters module 506 are conveyed to a physician who may choose to program the IMD to deliver stimulation through the selected stimulation channel and to detect for events through the selected detection channel. In some embodiments, the selections by the parameters module 506 can be communicated to a programmer 116 or patient monitor 110 for automatic programming into the IMD.

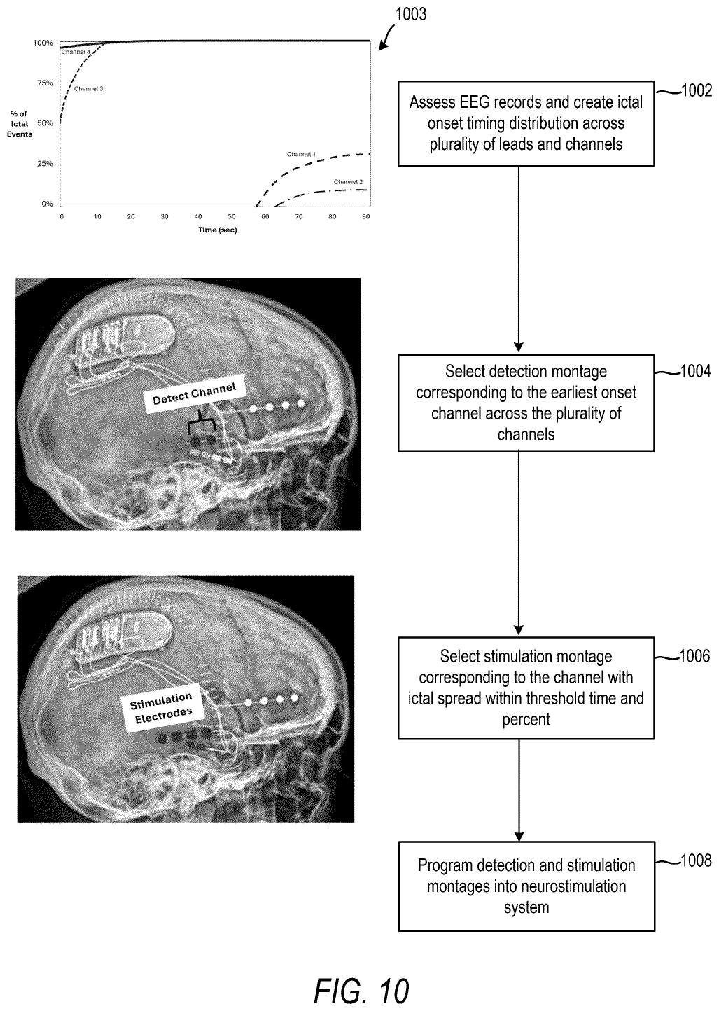

FIG. 10 is a flowchart of a method of determining one or both of a detection parameter and a stimulation parameter for a neurostimulation system based on electrographic data. The method invokes the principles of FIGS. 6-9 and can be implemented by the optimizer 104 of FIG. 5. While the flowchart of FIG. 10 and the following describes determining both of a detection parameter and a stimulation parameter, the method may include determining only one of a detection parameter and a stimulation parameter.

At block 1002, EEG records are processed by the electrographic events module 502 to identify EEG records having a specified event and to determine the onset times of such events. In some embodiments the event of interest is an ictal event and the event feature is a seizure onset. In some embodiments the event of interest is a sub-ictal event and the event feature is a Boolean combination of one or more of a half-wave detection and a line length detection. Additional disclosure on half-wave detections and line length detections and Boolean combinations are disclosed in U.S. Pat. No. 10,321,866, which is incorporated by reference. This information is then processed by the probabilistic module 504 to calculate a probabilistic distribution 1003 of event onset timing across a plurality of leads and detection channels.

At block 1004, a detection montage is optimized by the parameters module 506 based on the earliest event onset across all the events and channels. Detection montage refers to the configuration controlling which channel or channels is used for event detection, and consequently which electrodes are used for event detection. To this end, the parameters module 506 is configured to select as a detection channel, the channel that has the earliest onset of the event across the plurality of channels.

At block 1006, the stimulation montage is optimized by the parameters module 506 based on threshold criteria such as the observed spread with a set time frame. To this end, the parameters module 506 selects for stimulation the electrodes making up the channel having the earliest onset of the event across all channels. The parameters module 506 may additionally or alternatively select electrodes making up the channel or channels having an ictal spread within a threshold time and percent (e.g., with spread within 10 seconds, relative to the earliest onset of the event, over 50% of the samples).

At block 1008, the detection montage and the stimulation montage selected by the parameters module 506 are programmed in an implanted neurostimulation system 102. In some embodiments the programming is automatically performed by a programmer or patient monitor. The implanted neurostimulation system 102 subsequently detects the event through the detection montage and delivers stimulation therapy through the stimulation montage.

FIG. 11 is a flowchart of a method of determining one or both of a detection parameter and a stimulation parameter for a neurostimulation system based on electrographic data and imaging data. In this method, optimizer 104 is configured to select one or more settings for delivering stimulation therapy by the implanted medical device using a procedure that includes: estimating a target region (block 1102) of a brain for the detection of an event of interest and the delivery of neurostimulation therapy; refining a target region (block 1104); estimating stimulation settings (block 1106); and refining stimulation settings (block 1108).

In some embodiments, one or more steps of this procedure may be omitted. For example, an estimated target region may be used without refinement; an algorithm for target refinement may be used with no a priori estimated target region, or a generic estimated target region such as the entire brain; estimated stimulation settings may be used without refinement; and/or an algorithm for refinement of stimulation settings may be used with no initial estimate of stimulation settings, or a generic estimate of stimulation settings.

As disclosed above, the stimulation settings selected by the parameters module 506 may include but are not limited to amplitude, pulse width, and/or stimulation montage. Again, stimulation montage refers to one or more settings which determine the electrode path(s) that stimulation is delivered through. In its most general form, stimulation montage is a specification of one or more electrode groups, where an electrode group comprises a set of one or more electrodes which serve jointly as the cathode for stimulation and a set of one or more electrodes, distinct from the cathode set, which serve jointly as the anode for stimulation. Additionally, stimulation settings may include multiple amplitude and/or pulse width settings. For example, stimulation settings may include one amplitude setting for each electrode group in a stimulation montage, in an embodiment where the implantable neurostimulator 102 has multiple independent current sources available.

### Estimation of a Target Region (Block 1102)

In this step, the parameters module 506 determines an initial estimate of a target region of a brain for the detection of one or more events of interest and the delivery of neurostimulation therapy. An event of interest can be a clinical event, such a seizure, or a sub-clinical event, such as a sub-clinical electrographic seizure, or a sub-ictal or non-ictal epileptiform event. In some embodiments the initial estimate of a target region is determined based on a neuroanatomical atlas or on a neuroanatomical atlas and data (such as etiological data, seizure type, functional connectivity, metabolic, pre-or intra-operative electrophysiology, post-operative electrophysiology, or other clinical data) specific to the patient. In some embodiments the initial estimate of a target region is selected by a clinician or other individual via a graphical user interface and communicated to the parameters module 506. In some embodiments the initial estimate of a target region is determined via some combination of these methods or determined by other means.

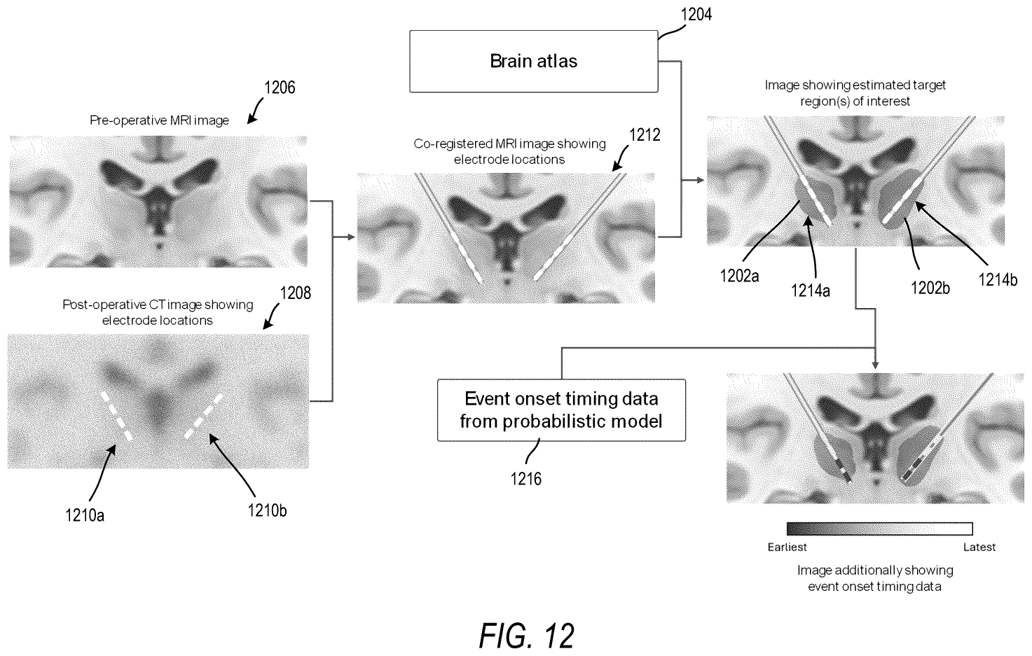

With reference to FIG. 12, in an example embodiment initial estimates of one or more target region 1202a, 1202b are determined automatically by the parameters module 506 using a brain atlas 1204 warped to the patient's own imaging data. Pre-operative MRI imaging 1206 is collected before the implant procedure; this is commonly done as part of surgical planning. After implant, post-operative CT imaging 1208 is collected to accurately show actual electrode locations 1210a, 1210b. The post-operative CT imaging 1208 is co-registered with pre-operative MRI imaging 1206 using techniques well-known in the art in order to generate a spatial model 1212 which shows electrode locations with respect to the patient's individual neuroanatomy. The spatial model 1212 is provided to the parameters module 506.

The brain atlas 1204 (for example, the Talairach atlas, MNI atlas, Desikan-Killiany atlas, Harvard-Oxford atlas, or other atlas), which is a comprehensive map of the typical human brain, is warped to - that is, transformed to bring into registration with - the patient's individual neuroanatomy by the parameters module 506 using techniques well-known in the art. This transformation allows brain structures 1214a, 1214b which are defined in the brain atlas 1204 to be identified, at least approximately, in the patient's own neuroanatomy and in the patient's own geometric coordinates. Based on these brain structures 1214a, 1214b, one or more initial estimated target regions 1202a, 1202b are determined by the parameters module 506.

In an example embodiment, the initial estimated target region is automatically determined by the parameters module 506 to be the centromedian nucleus of the thalamus as defined by said atlas. In another example embodiment, the initial estimated target region is automatically determined by the parameters module 506 to be the hippocampus as defined by said atlas.

In another example embodiment, a structure of interest, such as the hippocampus, is displayed on a computer interface. Using the interface, a user such as a physician or surgeon adjusts the boundaries of the structure to yield the initial estimated target region and this region is communicated to the parameters module 506.

In another example embodiment, a structure of interest (such as a neocortical, cortical, or subcortical seizure focus, or a thalamic nucleus) is defined or drawn by a user with the aid of a computer interface, serving as the initial estimated target region and this region is communicated to the parameters module 506.

Note that in these embodiments, the geometric data describing the initial estimated target region can be defined using the patient's own geometric coordinates, allowing further calculations to happen in “patient space” rather than “atlas space.”

In some embodiments, event onset timing data 1216 determined by the probabilistic module 504 is used by the parameters module 506 to select one or both of a detection channel and a stimulation channel, as described above with reference to FIG. 10.

### Refinement of a Target Region (Block 1104)

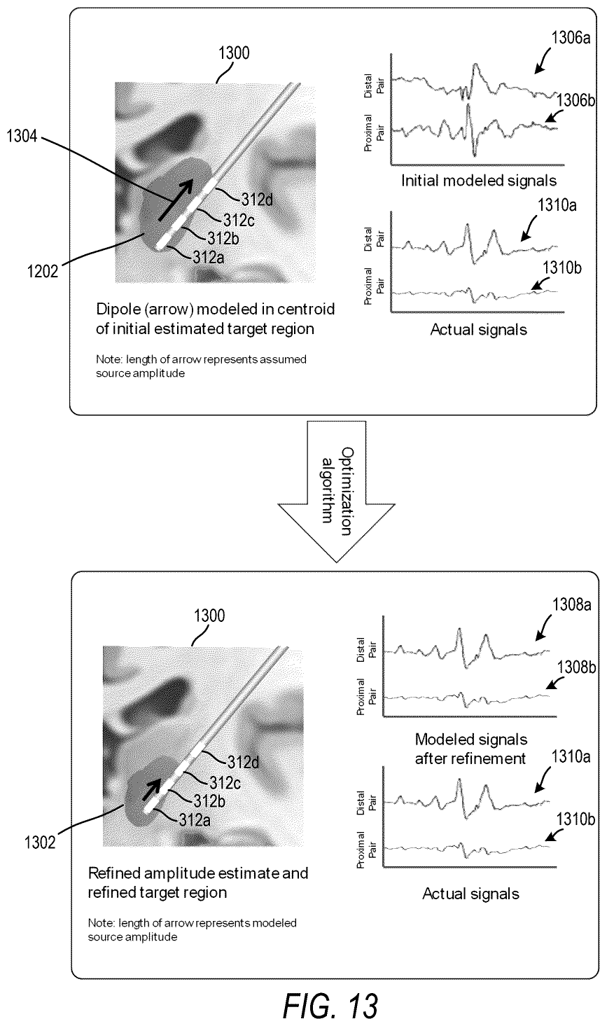

With reference to FIG. 13, in this step the parameters module 506 refines the initial estimated target region 1202a, 1202b using available electrographic data. In summary, the target region is adjusted, starting with the initial estimated target region 1202 (if available), based on an assessment of what spatial relationship between a target region and electrodes 312a-d would yield real, actual observed electrophysiological biosignals 1310a, 1310b. In some embodiments, this refinement includes creation of an electrical model based on the patient's neuroanatomy, such as a finite element model, to allow prediction of voltage waveforms 1306a, 1306b observed at electrodes given an assumed waveform at the target region 1202a, 1202b. In some embodiments, this refinement additionally or alternatively includes creation of a functional connectivity model based on the patient's neuroanatomy to allow prediction of phase offsets and/or arrival times observed at electrodes given assumed neural activity at the target region. In some embodiments, derived signals such as ensemble averages from many electrophysiological events may be used as actual biosignals 1310a, 1310b.

Continuing with FIG. 13, a finite element electrical model based on the patient's neuroanatomy is created by segmenting the patient's pre-operative MRI imaging data 1206 into a number of segments 1300 and assigning impedance properties to each segment, according to modeling techniques well-known in the art. Then, a dipole 1304 source is simulated at the centroid of the initial estimated target region 1202, with the dipole orientation aligned to the most dominant somatodendritic orientation within the structure, as determined by known neuroanatomical properties (for example, perpendicular to the plane of the pyramidal cell layer in neocortex or hippocampus).

An initial assumed current amplitude is assigned to the dipole source (for example, 10-300 nA-m) and the resulting voltages at electrodes 312a-d are calculated using forward modeling techniques well-known in the art (e.g., minimum-norm estimates) to produce initial modeled signals 1306a, 1306b. Using optimization algorithms well-known in the art (e.g., simulated annealing, beamforming, FOCUSS, or others), and regularizing solutions by preferring regions near the initial estimated target region 1202 (e.g., within 1 cm from the initial target estimate), a refined assumed current amplitude and refined target region 1302 may be determined having the greatest likelihood of yielding refined modeled signals 1308a, 1308b having a morphology, e.g., signal amplitudes and polarities, within a similarity threshold criterion of the actual signals 1310a, 1310b observed at electrodes 312a-d during real events.

In some embodiments, current amplitude is not assumed at the dipole source; instead or additionally, the current waveform generated at the source is allowed to be a free parameter which is estimated as part of the optimization process. A similarity criterion for such an optimization process may be, for example, arrival at a local minimum in the search space with respect to a weighted sum of (a) geometric distance between the initial estimated target region 1202 and refined target region 1302, and (b) Euclidean distance or dynamic time warping distance between the refined modeled signals 1308a, 1308b and the actual signals 1310a, 1310b.

This refined target region 1302 may be displayed on an interface for the user and/or used in subsequent steps to assist in estimation or refinement of stimulation settings. In some embodiments, the step of refining the target region may include assumption of a dipole distribution across the spatial extent of the target region, or assumption of a pseudomonopolar source at the centroid of the target region. In some embodiments, the step of refining the target region may comprise selecting the point within an initial estimated target region 1202 which is closest to the electrodes 312a-d forming the channel with the greatest likelihood of initial detection of an electrographic event, such as an epileptiform or ictal event. In some embodiments, the step of refining the target region may comprise selecting the point within an initial estimated target region 1202 which is closest to the electrodes 312a-d forming the channel with greatest signal amplitude during an electrographic event, such as an epileptiform or ictal event. In some embodiments, the step of refining the target region may additionally or alternatively include modeling of signal propagation delays along neural pathways, yielding a refined target region 1302 having a relatively high likelihood of yielding the signal timing and/or phase offsets present in actual signals 1310a, 1310b observed at electrodes 312a-d, during real events.

While the foregoing describes the initial estimation and/or refinement of a target region of the brain for a pathological event of interest, the system can also determine optimal target regions of the brain for other pathological events. For example, the system can determine a first optimal target region for a pathological event corresponding to a seizure with neocortical onset, and a second target region for a pathological event corresponding to a seizure with mesial temporal onset.

### Estimation of Stimulation Settings (Block 1106)

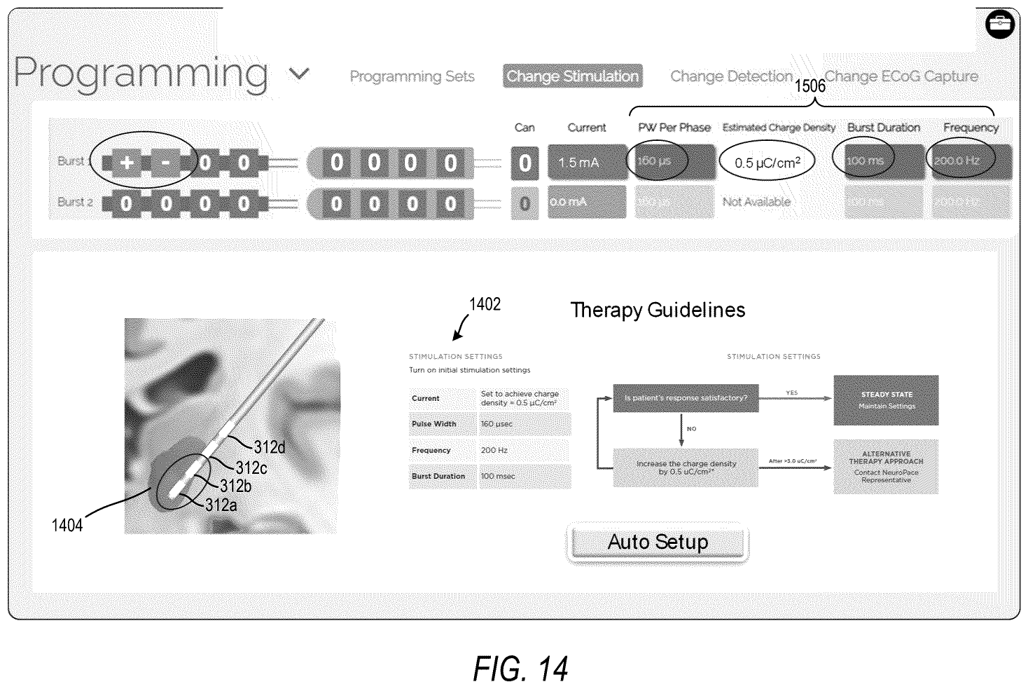

With reference to FIG. 14, having determined at target region 1404 of the brain, which may be the initial estimated target region 1202 or refined estimated target region 1302, for one or more events of interest, the parameters module 506 determines one or more additional aspects of the neuromodulation therapy. For instance, one or more initial estimates of stimulation settings 1402 or parameter sets that optimize the effect of neuromodulation therapy delivered by the device can be determined. This initial estimate of stimulation settings 1402 may be determined by the parameters module 506 based on predetermined therapy guidelines. In other embodiments the initial estimate of stimulation settings 1402 may be selected by a clinician or other user using a graphical user interface and communicated to the parameters module 506. In other embodiments, the initial estimate of stimulation settings 1402 is recommended by an automated proposing system such as a deep learning model, and/or determined by other means.

In other embodiments, the initial estimated stimulation settings 1402 may be selected by an algorithm which adapts predetermined therapy guidelines using the target region 1404 as described above. With continued reference to FIG. 14, in such an example embodiment the initial estimated stimulation settings of a target region 1404 is determined as follows. Pre-existing therapy guidelines are implemented or programmed into the optimizer 104. Examples of such guidelines include: “for a depth lead located in the hippocampus, begin with bipolar stimulation across adjacent electrodes on the lead, with 160μs pulse width, 100 ms burst duration, 200 Hz pulse frequency, and amplitude sufficient to yield charge density 0.5μC/cm²”, or “for a cortical strip lead located near a focal cortical onset, begin with monopolar cathodal stimulation referenced to the neurostimulator case, with 160 μs pulse width, 100 ms burst duration, 200 Hz pulse frequency, and amplitude sufficient to yield charge density 0.5μC/cm².”

The relevant guideline is then adapted by the parameters module 506. For instance, the parameters module 506 is configured to assign the initial estimated stimulation settings 1402 recommended by the guideline to the pair of electrodes 312a, 312b closest to the centroid of the target region 1404. Alternatively, the initial estimated stimulation settings 1402 can be assigned by the parameters module 506 to the set of electrodes within, substantially within, or touching the target region 1404, or to the set of electrodes within a certain distance from the target region (e.g., within 5 mm from the target region).

In another embodiment, initial estimated stimulation settings 1402 are generated by assigning the stimulation settings recommended by the guideline to the electrodes 312a-d forming the channel with the greatest likelihood of initial detection of an electrographic event, such as an epileptiform or ictal event. In another embodiment, initial estimated stimulation settings 1402 are generated by assigning the stimulation settings recommended by the guideline to the electrodes 312a-d forming the channel with the highest signal amplitude associated with an electrographic event, and/or the earliest phase offset or signal timing associated with an electrographic event.

The initial estimated stimulation settings 1402 then may be displayed to and/or offered for adjustment to the user via a graphical user interface, and/or made available for programming or programmed to the implanted neurostimulation system 102, and/or further refined as described below.

### Refinement of Stimulation Settings (Block 1108)

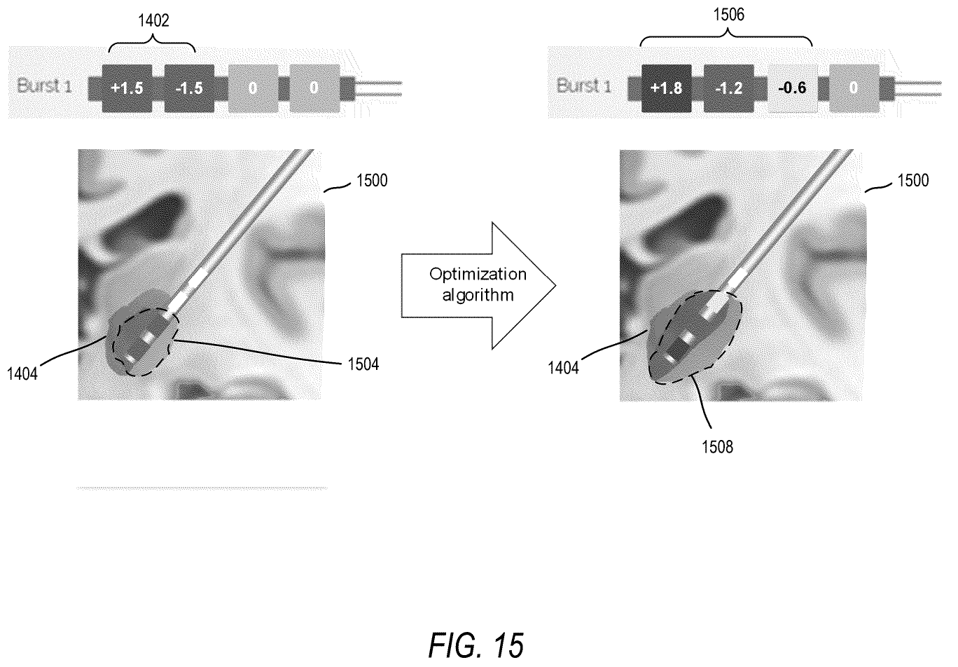

In this step, the parameters module 506 refines the initial estimated stimulation settings 1402 by modeling the effect of stimulation settings on the target region 1404. With reference to FIG. 15, in an example embodiment a finite element electrical model based on the patient's neuroanatomy is created by segmenting the patient's pre-operative MRI imaging data 1206 into a number of segments 1500 and assigning impedance properties to each segment, according to modeling techniques well-known in the art; or, if such a model was created in a previous step, this model is re-used.

Then, given initial estimated stimulation settings 1402, the electrical field strength 1504 and/or current density at the target region 1404 is calculated using forward modeling techniques well-known in the art (e.g., minimum-norm estimates). Using optimization and regularization algorithms well-known in the art (e.g., simulated annealing, beamforming, FOCUSS, or others), refined stimulation settings 1506 may be determined having a desired voltage gradient (e.g., 10 V/mm) or a certain predicted neuromodulatory effect (e.g., 50% of pyramidal cells activated) at the target region 1404, which yield a predicted neuromodulatory region 1508 more closely aligned with the target region 1404, and which comply with technological constraints of the implantable neuromodulation system (e.g., current limits and number of independent current channels available). A similarity criterion for such an optimization process may be, for example, arrival at a local minimum in the search space with respect to a weighted sum of (a) voxels in the target region 1404 not also within the predicted neuromodulatory region 1508 and (b) voxels in the predicted neuromodulatory region 1508 not also within the target region 1404.

The region in which the voltage gradient due to stimulation is above a desired threshold (e. g, 10 V/mm) or yields a desired neuromodulatory effect may be displayed via a graphical user interface. In some embodiments, the user may further refine the stimulation settings 1506 while observing the predicted effect of the settings on predicted neuromodulatory region 1508, voltage gradient, or electrical field. In some embodiments, the user may refine the stimulation settings manually using this interface, without the need for an electrical model or search algorithm. The initial estimated stimulation settings 1402 then may be displayed to and/or offered for adjustment to the user via a graphical user interface, and/or made available for programming or programmed to the implanted neurostimulation system 102, and/or further refined as described below.

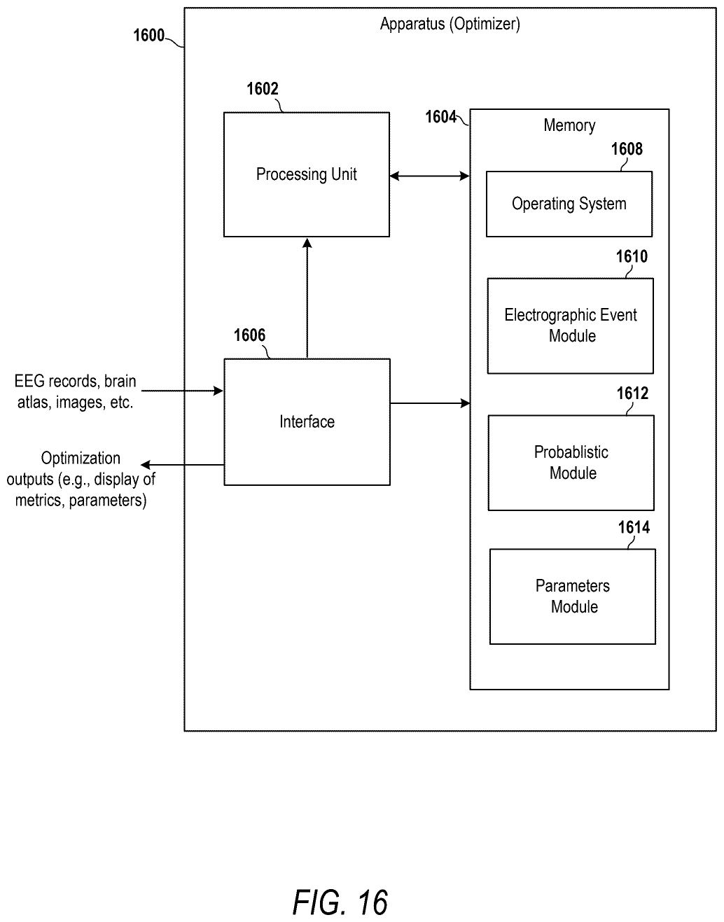

FIG. 16 is a schematic block diagram of an apparatus 1600 corresponding to the optimizer 104 of FIG. 1. The apparatus 1600 is configured to execute instructions related to the optimizer processes described above with reference to FIGS. 1-15. The apparatus 1600 may be embodied in any number of processor-driven devices, including, but not limited to, a server computer, a personal computer, one or more networked computing devices, an application-specific circuit, a minicomputer, a microcontroller, and/or any other processor-based device and/or combination of devices.

The apparatus 1600 may include one or more processing units 1602 configured to access and execute computer-executable instructions stored in memory 1604. The processing unit 1602 may be implemented as appropriate in hardware, software, firmware, or combinations thereof. Software or firmware implementations of the processing unit 1602 may include computer-executable or machine-executable instructions written in any suitable programming language to perform the various functions described herein. The processing unit 1602 may include, without limitation, a central processing unit (CPU), a digital signal processor (DSP), a microprocessor, a microcontroller, or any combination thereof.

The memory 1604 may include, but is not limited to, random access memory (RAM), flash RAM, magnetic media storage, optical media storage, and so forth. The memory 1604 may include volatile memory configured to store information when supplied with power and/or non-volatile memory configured to store information even when not supplied with power. The memory 1604 may store various program modules, application programs, and so forth that may include computer-executable instructions that upon execution by the processing unit 1602 may cause various operations to be performed. The memory 1604 may further store a variety of data manipulated and/or generated during execution of computer-executable instructions by the processing unit 1602.

The apparatus 1600 may further include one or more interfaces 1606 that may facilitate communication between the apparatus and one or more other apparatuses. For example, the interface 1606 may be configured to receive EEG records from a database and to output information, e.g., therapy suggestions, to a display. Communication may be implemented using any suitable structure or communications standard, wired or unwired. For example, communication with a database may be through a LAN interface that implement protocols and/or algorithms that comply with various communication standards of the Institute of Electrical and Electronics Engineers (IEEE), such as IEEE 802.11. Communication with a user interface and display may be through wired or unwired structures.

The memory 1604 may store various program modules, application programs, and so forth that may include computer-executable instructions that upon execution by the processing unit 1602 may cause various operations to be performed. For example, the memory 1604 may include an operating system module (O/S) 1608 that may be configured to manage hardware resources such as the interface 1606 and provide various services to applications executing on the apparatus 1600.

The memory 1604 stores additional program modules such as: (1) an electrographic event module 1610, (2) probabilistic module 1612; and (3) a parameters module 1614;. Each of these modules includes computer-executable instructions that when executed by the processing unit 1602 cause various operations to be performed, such as the operations described earlier with reference to FIGS. 1-15.

The various aspects of this disclosure are provided to enable one of ordinary skill in the art to practice the present invention. Thus, the claims are not intended to be limited to the various aspects of this disclosure, but are to be accorded the full scope consistent with the language of the claims. No claim element is to be construed under the provisions of 35 U.S. C. § 112, sixth paragraph, unless the element is expressly recited using the phrase “means for” or, in the case of a method claim, the element is recited using the phrase “step for.”

What is claimed is:

1. A system comprising:
a database comprising a plurality of records of biosignal activity of a brain sensed at different locations within or on the brain by different channels of an implanted medical device;
an electrographic event module configured to process the plurality of records to identify records corresponding to an event of interest, and for each identified record, to detect an event feature and a time of the event feature; and
a probabilistic module configured to process the times of the event feature for the identified records and to determine for the different locations within or on the brain, a metric of times the event feature was first detected at that location.

2. The system of claim 1, further comprising a parameters module configured to select a detection channel location from among the different locations for detecting by the implanted medical device, based on the metrics determined by the probabilistic module.

3. The system of claim 5, wherein the parameters module is further configured to provide information on the detection channel location to a programmer for programming into the implanted medical device.

4. The system of claim 3, wherein the information comprises an electrode montage of the implanted medical device that at least partially overlaps the detection channel location.

5. The system of claim 4, further comprising a parameters module configured to select a stimulation channel location from among the different locations for stimulation therapy delivery by the implanted medical device, based in the metrics determined by the probabilistic module.

6. The system of claim 5, wherein the parameters module is further configured to provide information on the stimulation channel location to a programmer for programming into the implanted medical device.

7. The system of claim 6, wherein the information comprises an electrode montage of the implanted medical device that at least partially overlaps the detection channel location.

8. The system of claim 1, wherein the event of interest is an ictal event and the event feature is a seizure onset.

9. The system of claim 1, wherein the event of interest is a sub-ictal event and the event feature is a Boolean combination of one or more of a half-wave detection and a line length detection.

10. The system of claim 1, wherein the probabilistic module is configured to provide information to a display that enables the display to display the different channels and different locations within or on the brain and to graphically convey the spatial and probabilistic nature of the events of interest relative to the different channels and the different locations.

11. An apparatus for selecting a therapy parameter of an implanted medical device, the apparatus comprising:
an interface configured to receive a plurality of records of biosignal activity of a brain sensed at different locations within or on the brain by different channels of an implanted medical device; and
a processor configured to:
process the plurality of records to identify records corresponding to an event of interest, and for each identified record, to detect an event feature and a time of the event feature;
process the times of the event feature for the identified records and determine for the different locations within or on the brain, a metric of times the event feature was first detected at that location; and
select, based on the metrics determined by the probabilistic module, one or both of a detection channel location from among the different locations for detecting by the implanted medical device and a stimulation channel location from among the different locations for stimulation therapy delivery by the implanted medical device.

12. A method of selecting one or more therapy parameters of a neurostimulation system, the method comprising:
identifying, from a set of EEG records captured through a plurality of channels of the neurostimulation system, those EEG records having a specified event;
for the identified EEG records, determining the onset time of the specified event;
calculating a probabilistic distribution of event onset timing across a plurality of channels;
selecting at least one therapy setting based on the probabilistic distribution;
programming the therapy setting into the neurostimulation system; and
operating the neurostimulation system based on the therapy setting.

13. The method of claim 12, wherein:
selecting at least one therapy setting comprises selecting as a detection montage, the channel that has the earliest onset of the event across the plurality of channels; and
operating the neurostimulation system based on the therapy setting comprise detecting the event through the detection montage.

14. The method of claim 12, wherein:
selecting at least one therapy setting comprises selecting as a stimulation montage from among a plurality of stimulation montages of the neurostimulation system, the stimulation montage that meets a criterion; and
operating the neurostimulation system based on the therapy setting comprises delivering stimulation therapy through the stimulation montage.

15. The method of claim 14, wherein the specified event is an electrographic seizure and the criterion is a seizure spread within a threshold time and percent.

16. A system for selecting therapy parameters for a neurostimulation system associated with a brain, the system comprising:
a device configured to sense biosignal activity from the brain; and
an optimizer configured to:
select a target region of the brain for neuromodulation therapy, which target region minimizes the difference between real biosignals captured by the device and modeled biosignals; and
determine stimulation settings for neuromodulation therapy, which stimulation settings result in a modeled electrical characteristic of stimulation at a target region that satisfies a criterion.

17. The system of claim 16, wherein the optimizer is configured to select a target region of the brain by being further configured to:
a. select an initial estimated target region characterized by a shape and size;
b. create a modeled biosignal for a detection montage of the neurostimulation system that at least partially overlaps the initial estimated target region
c. obtain a real biosignal captured in the estimated target region by the detection montage;
d. determine if the modeled biosignal satisfies a similarity criterion relative to the real biosignal; and
e. if the similarity criterion is not satisfied, refine the estimated target region and repeat steps b, c, and d until the modeled biosignal is similar to the real biosignal.

18. The system of claim 17, wherein the similarities criterion is a measure of morphology similarity between the modeled biosignal and the real biosignal.

19. The system of claim 17, wherein the refined estimated target region is characterized by at least one of a shape and a size different from those of the initial estimated target region.

20. The system of claim 16, wherein the optimizer is configured to select stimulation settings by being further configured to:
a. select an initial stimulation parameter;
b. determine a modeled electrical characteristic of the initial estimated target region that would result from delivery of stimulation based on stimulation settings comprising the initial stimulation parameter and an initial stimulation montage of the neurostimulation system;
c. determine if the modeled electrical characteristic satisfies a criterion; and
d. if the criterion is not satisfied, refine the stimulation settings and repeat steps b and c until the criterion is satisfied.

21. The system of claim 20, wherein the refined stimulation setting comprises one or both of a refined stimulation parameter different from the initial stimulation parameter, and a refined stimulation montage different from the initial stimulation montage.

22. The system of claim 16, wherein the optimizer is configured to communicate information corresponding to the determined stimulation settings for programming into the neurostimulation system.

23. A method for selecting one or more settings for delivering stimulation therapy by a neurostimulation system, the method comprising:
estimating a target region of a brain for the detection of an event of interest and the delivery of neurostimulation therapy; and
estimating stimulation settings for the delivery of the neurostimulation therapy.

24. The method of claim 23, wherein estimating a target region comprises:
generating a spatial model that includes brain structures and electrodes of the neurostimulation system based on imaging data; and
selecting as an estimated target region, a brain structure of interest in the spatial model.

25. The method of claim 24, further comprising:
a. determining a modeled biosignal for a detection montage;
b. determining a real biosignal based on EEG records of the detection montage;
c. determining if the modeled biosignal satisfies a similarity criterion relative to the real biosignal; and
d. if the similarity criterion is not satisfied, refining the estimated target region and repeating steps a, b, and c until the modeled biosignal is similar to the real biosignal.

26. The method of claim 24, wherein estimating stimulation settings comprises:
obtaining initial stimulation parameters from a therapy guideline.

27. The method of claim 26, further comprising:
a. determining a modeled electrical characteristic of the initial estimated target region that would result from delivery of stimulation based on the initial stimulation settings comprising the initial stimulation parameters and the stimulation montage;
b. determine if the modeled electrical characteristic satisfies a criterion; and
c. if the criterion is not satisfied, refine the stimulation settings and repeat steps a and b until the criterion is satisfied.
